(19) United States
(12) Patent Application Publication
(10) Pub. No.: US 2015/0265237 A1
(43) Pub. Date: Sep. 24, 2015

(54) METHOD AND DEVICE FOR GENERATING A THREE-DIMENSIONAL IMAGE OF AN OBJECT

(71) Applicants: FRAUNHOFER-GESELLSCHAFT ZUR FÖRDERUNG DER ANGEWANDTEN FORSCHUNG E.V., München (DE); CHARITÉ - UNIVERSITÄTSMEDIZIN BERLIN, Berlin (DE)

(72) Inventors: Erwin Keeve, Potsdam (DE); Marc Käseberg, Biesenthal (DE); Fabian Stopp, Neuenhagen (DE); Eckart Uhlmann, Kiebitzreihe (DE); Sebastian Engel, Munster (DE)

(21) Appl. No.: 14/437,656

(22) PCT Filed: Oct. 21, 2013
(86) PCT No.: PCT/EP2013/071954

§ 371 Date: Apr. 22, 2015

(30) Foreign Application Priority Data
Oct. 22, 2012 (DE) 10 2012 219 269.7

(51) Int. Cl. A61B 6/00; A61B 6/02; A61B 6/14; G06T 11/00

(52) U.S. Cl. CPC A61B 6/5223; G06T 11/005; G06T 11/008; A61B 6/022; A61B 6/14; A61B 6/5252; G06T 2211/416

(57) ABSTRACT

The invention relates to a method and a device for generating a three-dimensional image of an object, said device comprising a movable radiation source, a radiation detector, and an analyzing unit. The radiation source is moved relative to the object to be imaged into multiple positions in a first movement on a first track which lies on a first plane in order to capture images, at least one image being captured in each said position. The three-dimensional image is reconstructed from the captured images by the analyzing unit. The radiation source and the radiation detector carry out a second movement relative to the object to be imaged on a second track which is at least partly different from the first track at the same time as the first movement in order to reduce artifacts in the image, said artifacts being generated due to radiation absorption. In the process, the first movement and the second movement are superimposed.

The present invention relates to a method as well as to a device for producing a three-dimensional image of an object. The present invention moreover relates to a computer program product, which contains a command sequence which activates a device for producing a three-dimensional image of an object.

Imaging devices such as X-ray systems for example, in the field of medicine serve for examining patients, wherein a three-dimensional image can be reconstructed from two-dimensionally recorded pictures by way of a computation method, with special embodiments of the respective imaging device.

Thus with digital volume tomography (DVT) with a volume tomograph for example, several pictures are recorded by way of an X-ray tube which rotates about the patient and by way of a sensor which lies opposite the X-ray tube during the rotation, and these pictures are subsequently processed into a three-dimensional reconstruction of a scanned region. Digital volume tomography as a medical imaging system is applied largely for three-dimensional picture recording of the skull. The method has a significant artefact formation with materials which absorb X-rays to a great extent, for example such as metal implants, tooth fillings or tooth braces, since the digital tomograph moves the X-ray source and the sensor lying opposite this, mostly in a horizontal manner on a circular path. These materials delete information on tissue which is located in front of or behind this, in two-dimensional pictures or at least greatly reduce this information. This leads to structures in the reconstructed three-dimensional picture which do not correspond to the spatial distributions of the tissue to be examined, in a picture plane, the so-called metal artefacts.

Methods and devices, by way of which these artefacts can be reduced for diagnostic validity, are already known from the state of the art. Solution ideas for this until now have been based on algorithmic adaptations in a data processing before, during or after reconstruction of the three-dimensional image. The document DE 103 20 233 A1 for example discloses a computation of artefact projection pictures on the basis of a provisional, three-dimensional reconstruction and metallic parts which are detected therein. These artefact projection pictures are subsequently used, in order to improve the original two-dimensional projection pictures, which finally leads to a reduction of the metal artefacts in the final three-dimensional picture data. A general disadvantage of such algorithmic solutions however is the fact that correction methods attempt to reduce the effects of an information loss by way of an absorption of the metal parts, or attempt to recreate/retrieve the lost data by way of interpolation or extrapolation, wherein the data which is deleted during the recording however cannot be completely reconstructed.

It is therefore the object of the present invention, to develop a method and a device, with which the mentioned disadvantages are avoided, with which thus a reduction of artefacts with a three dimensional image of an object is effected in a simple and rapid manner, without subsequent algorithmic processing of recorded pictures.

According to the invention, this object is achieved by a method according to claim 1, a device according to claim 14, as well as a computer program product according to claim 18, for the control of a device or for carrying out a method. Advantageous further developments are described in the dependent claims.

A method according to the invention for producing a three-dimensional image of an object makes use of an imaging device. The imaging device comprises a movable beam source, a beam detector and an evaluation unit, for recording pictures of the object to be imaged from several positions. The beam source, for recording a picture is moved relative to the object to be imaged, in a first movement on a first path lying in a first plane into several positions. At least one picture is recorded in the positions in each case, wherein the three-dimensional image is reconstructed from the recorded pictures by way of the evaluation unit. The beam source however at the same time as the first movement carries out a second movement relative to the object to be imaged, on a second path which at least in sections is different to the first path, for reducing an artefact in the image, said artefact being produced by shadowing. The first movement and the second movement hereby superimpose. A distance between the beam source and the object to be imaged is constant during the superposition of the first movement and the second movement, thus during the movement of the beam source.

Additional projection perspectives are generated by way of the superposition of the first movement and the second movement, which has the effect that these are carried out simultaneously, and by way of the recording of pictures at different positions. A greater picture quality and an improved technical usability are achieved by way of supplementing the first movement with the second movement, i.e. a superposition with at least one further movement or at least one further degree of freedom. The new projection perspectives contain projection information or picture information on the object to be imaged, which would not be able to be acquired by way of picture recording solely along the first path. Moreover, a simple geometric linking or relation simplifying the evaluation is given due to the constant distance between the beam source and the object to be imaged. In particular, these additional projection perspectives have information which would have been deleted with a conventional picture recording, on account of metal components, such as dental implants or tooth fillings for example, on examining a skull. An exact representation of the scanned object is possible on the basis of the additionally obtained information, without having to carry out a separate algorithm for this, for reducing the artefacts. A speed, at which the imaging method is carried out, is increased by way of this, and a quality of the obtained picture data is also improved. The advantages of the geometry of the first path as well as that of the second path can be utilised by way of the movement of the beam source on a combined path which results from the first path and the second path. Thus two movements are carried out during the recording, and these lead to different recording positions and new projection perspectives. The combined path, on which the superimposed movement takes place, hereby describes the movement of the beam source in space. A path in this context is to be understood as a combination of points in space, through which the beam source successively runs or would run without superposition. The combined path is thus indeed the trajectory of the movement which is composed of the individual paints which are run through during the recording.

At least a part of the object to be imaged, preferably a middle point of the object to be imaged can lie in an axis intersection point of a first axis, about which the first movement is effected, and of a second axis, about which the second movement is effected. The middle point can be a geometric middle point as well as a centre of gravity. It is thus ensured that the first movement as well as the second movement runs around the target region which is indeed to be examined, is of interest and is spatially defined by the middle point, and thus picture information on this target region is available at different angles. Herewith, a simple further processing of the obtained picture data is simultaneously rendered possible. A distance between the beam source and the middle point and/or a distance between the beam detector and the middle point is preferably constant during the first movement and the second movement.

Typically, the first axis is perpendicular to the first plane and/or the first axis and the second axis are perpendicular to one another, so that a particularly clear geometric relation between the first movement and the second movement is given, and an accordingly simplified further processing can be carried out. In particular, the first axis can also lie completely in a second plane of the movement, and/or the second axis can lie completely in the first plane. Moreover, one can envisage a middle axis running from a beam bundle emitted from the beam source through the axis intersection point, so that the region around this axis intersection point is illuminated/irradiated with a sufficient high intensity and one images in a corresponding quality.

The distance between the beam source and the object to be imaged can be defined as a distance between a geometric middle point or a centre of gravity of the beam source and the geometric middle point or the centre of gravity of the object to be imaged. However, one can also envisage using a reference point on the surface for determining the distance, instead of the geometric middle point or the centre of gravity of the object to be imaged, wherein preferably a point of the surface which lies closest to the beam source serves as a reference point.

The distance between the beam source and the object to be imaged and the distance between the beam detector and the object to be imaged are typically equally large, in order to have a simple geometric relation between the beam source, the object to be imaged, and the beam detector. An evaluation is simplified by way of this. Alternatively, the distance between the beam source and the object to be imaged, typically the middle point of the object to be imaged, can also be smaller than the distance between the beam detector and the object to be imaged, here too typically the middle point of the object to be imaged.

The second movement is typically effected at least in sections in one of two half-spaces which are defined by the first plane. The first plane thus divides the space surrounding the object to be imaged into two half-spaces, so that the second movement for example can only be effected in one of the two half-spaces, thus in particular exclusively below or above, or to the left or to the right of the first plane, or the second movement is effected in both half-spaces, wherein one runs through the first plane at certain time periods during the second movement. Thus a recording of pictures in a multitude of positions is possible and the movement can be individually set, depending on the desired field of application. Preferably, the second movement is however effected at least in sections in both half-spaces and/or the second movement at least in sections is a periodic movement, particularly preferably a sinusoidal movement or one effected in discrete steps. In particular, the second movement can also consist completely of a periodic movement. A further processing of the obtained data is significantly simplified by way of a periodicity of the respective movement. The second movement can of course also follow a freely selectable course. A setting of the second movement is also possible depending on the geometry of the object to be imaged.

Typically, the first movement is a circular movement, an elliptical movement or a movement which is freely selectable in its course, and/or the second movement an inclination movement. The first movement, in particular the circular movement can preferably be effected horizontally or vertically, for example with a seated or lying patient or object. The first path can moreover be a closed path, i.e. a path with which a starting point coincides with an end point. Accordingly the first movement can be a closed movement, with which the starting point corresponds to the end point. The object to be imaged is typically completely travelled around at least one by the first movement by way of this, so that picture information is available from perspectives all around the object to be imaged. Of course, one can also envisage using an open path, thus one which is not closed, for the first path, which is to say carrying out an open movement.

The perspective is changed in each case and additional picture information is made available by way of the inclination of the inclination movement. Typically, at least the beam source is moved in the first movement on the first path, in particular in the circular movement on a circular path, about at least 90°, preferably at least 180°, particularly preferably about at least 360° . The beam source alternatively or additionally in the inclination movement can at least be inclined by maximally between 1° and 45°, preferably maximally between 10° and 35°, particularly preferably maximally between 15° and 30°, with respect to a plane of the first movement. Pictures from a plurality of perspectives are possible without the beam source having to be moved too greatly, due to the specified angle ranges.

The path of the first movement and the path of the second movement can be adapted to the object to be examined, in order to achieve an optimal reconstruction volume and/or an as high as possible spatial resolution.

The beam detector is typically co-moved with the movement of the beam source, but it is also possible to keep the beam detector spatially fixed and to only move the beam source. It is likewise also possible for the beam detector to also carry out the first movement and the second movement of the beam source or to undergo only one of the two movements together with the beam source. The beam detector is hereby typically only led in the circular movement, whereas it does not undergo the inclination movement, but of course it can also participate only on the inclination movement and not undergo the circular movement. The beam detector is preferably movable independently of the beam source.

It is also possible for the inclination angle about the inclination axis, thus about the second axis, to be variable, thus not constant, during the rotation movement or circular movement about the first axis, i.e. about the rotation axis. It is likewise possible to select positions for a picture recording which are adapted to the geometry of the object to be imaged on account of this.

One can further envisage carrying out the first movement and/or the second movement driven in an automated manner at least in sections and/or in a manual manner at least in sections. The setting of the positions used for the picture recording can thus be predefined and be moved to or be directly set according to the wishes of the user, without any effort on the part of the user. Typically, a speed of the first movement and/or a speed of the second movement are constant or variable in sections. The speed of the movements is preferably an angular speed. This permits the method to be carried out more rapidly since the angular speed can also be adapted to a number of pictures which are to be recorded in the respective positions. The angular speed of the second movement at least in sections can preferably always be higher that the angular speed of the first movement. A lowering, thus a slowing down of the angular speed of the first movement is particularly preferably effected on increasing the angular speed of the second movement, thus an acceleration, in order to ensure a high accuracy of the recording due to the reduced acceleration forces. Conversely, an increase of the angular speed of the first movement can be effected given a reduction of the angular speed of the second movement. One obtains many recordings at different inclination angles, of regions which are of particular interest on account of this, whereas a comparatively low angular range of the rotation movement is swept over in the same time.

The beam source is preferably moved on a virtual sphere surface, wherein a middle point of the sphere coincides with the reference point which on the object side defines the distance between the beam source and the object to be imaged. The mentioned distance is always kept constant by way of this and a simple computation of the moment in spherical coordinates is rendered possible by way of this. The beam detector can additionally also be moved on the sphere surface or alternatively on a sphere surface of a sphere with a smaller or larger radius.

The first movement is typically carried out at lower frequency than the second movement, but however both movements can also be carried out at the same frequency, or the first movement carried out at a higher frequency than the second movement. A scanning according to the demands can be achieved in a simple manner by way of a selection of the frequency, and thus artefacts in the images can be efficiently reduced on account of the more frequent recording of regions which were covered in the previous recordings. The first path and the second path can alternatively or additionally be different to one another in sections.

Preferably, the frequencies of the first movement and of the second movement have a defined ratio to one another. This ratio is typically at least or precisely 1:2, 1:3 or 1:4, i.e. the second movement runs precisely with double, threefold or fourfold the frequency of the first movement, in order to obtain as many as possible different projections perspectives. Alternatively, with the previously mentioned conditions, it is possible for the first movement to have a greater frequency than the second movement.

Moreover, the evaluation unit can be configured to process all projection images into a three-dimensional projection image and hereby to automatically correct regions shaded by artefacts, by way of one or more recordings of the shaded region at a different recording angle. The evaluation unit moreover preferably comprises an output (issue) unit, on which the recorded individual pictures as well as alternatively or additionally the reconstructed three-dimensional image can be represented. The evaluation unit for this is typically configured to control the device.

A device for producing a three-dimensional image of an object comprises a beam source which is led in a first movement about the object to be imaged on a first path lying in a first plane, a beam detector and an evaluation unit. The beam source is movable relative to the object to be imaged, simultaneously with the first movement, by way of a second movement on a second path, for reducing an artefact of the obtained image which is produced by a shading. The first movement and the second movement hereby superimpose and a distance between the beam source and the object to be imaged is constant during the superposition of the first movement and the second movement. Thus several recordings of the object to be imaged can be made from different perspectives, so that additional picture information can be obtained by the additional projection perspectives. Preferably hereby, the first plane as well as the second plane runs through the object to be imaged.

The device is typically suitable for carrying out the already described method. One can further envisage the beam detector being movable, wherein the beam detector is preferably movable on the first path and/or the second path and/or the beam source and/or the beam detector can be inclined with respect to the plane of the first path and/or of the first movement, for carrying out the second movement. A variability of the device is increased by way of this, by way of the beam detector also carrying out at least one of the movements of the beam source. The beam source and the beam detector are preferably configured to always be located opposite one another. A clear geometric arrangement of the beam source and the beam detector to one another is rendered possible during the first movement as well as during the second movement, and this simplifies an evaluation of the obtained picture information. The beam source and the beam detector can have a rigid or flexible, i.e. non-rigid coupling to one another for this. This is preferably realised by a robot arm as a coupling which can be moved via several pivots attached on the robot arm.

The first path and/or the second path of the beam source and/or of the beam detector can be set by a linear pivot, a rotation pivot and/or a motorised joint arm. With this, the first movement and the second movement are also set accordingly. A joint arm is thereby to be understood as an arm with at least one joint, with which at least one rotation about an axis can be carried out via a joint. Typically, the joint arm can be adjusted only rotarily and not translatorily. The pivots are typically arranged directly on the beam source or on the beam detector, so that a torque is minimised with the movement of the rotation source or beam detector, and further parts of the device do not need to be co-moved.

Typically, the device is an X-ray device with an X-ray source as a beam source and with an X-ray detector as a beam detector. The beam detector can be a flat detector with a scintillator layer. The artefacts in this case are produced by a shading of the X-ray beam which can also be given by a total absorption of the X-ray radiation. The evaluation unit is preferably realised by a computer with a display and can be configured to use an iterative reconstruction method and/or an adapted rear projection method.

A computer program product according to the invention comprises a command sequence which controls a device for producing a three-dimensional image of an object. The device comprises a beam source which is led in a first movement about the object to be imaged, on a first path lying in a first plane, a beam detector and an evaluation unit. The computer program product controls the device in a manner such that the beam source for the picture recording is moved relative to the object to be imaged, on the first path in the first movement into several positions, in which in each case at least one picture is recorded, wherein the three-dimensional image is reconstructed from the recorded pictures by way of the evaluation unit, and the beam source simultaneously to the first movement carries out a second movement relative to the object to be imaged, on a second path which is different from the first path at least in sections, for reducing an artefact in the image, said artefact being produced by a shading. The first movement and the second movement hereby superimpose and a distance between the beam source and the object to be imaged remains constant during the superposition of the first movement and the second movement. The computer program product is typically configured to activate the already described device and/or to carry out the previously described method.

The implementation of the method and/or the activation of the device by the computer program product are typically effected when the computer program product runs on a computation unit.

The computer program product can preferably be loaded directly into an internal memory of the computation unit or is already stored in this and typically comprises parts of a program code for carrying out the described method or for activating the described device when the computer program product runs or is carried out on the computation unit. The computer program product can be stored on a machine-readable carrier, preferably a digital memory medium. The computer program product can also comprise a computer program, which comprises software means for carrying out the described method and/or for activating the described device when the computer program is carried out in an automation system or on the computation unit.

The previously already described device, the already described method and/or the computer program product can be applied in medical applications, preferably within the framework of digital volume tomography or a three-dimensional imaging based on beaming-through, in dental medicine, oral surgery, jaw surgery, facial surgery, ears-nose-and-throat medicine and/or destruction-free industrial imaging.

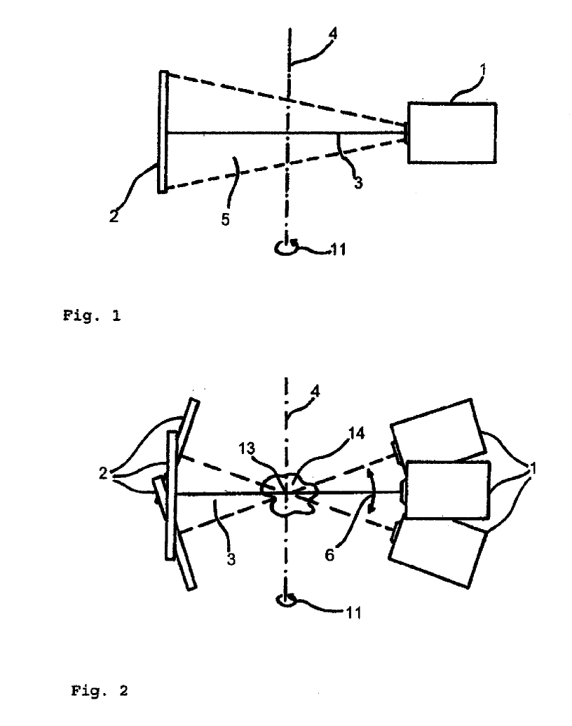

Embodiment examples of the invention are represented in the drawings and are explained hereinafter by way of FIGS. 1-11.

There are shown in:

FIG. 1: a schematic lateral view of a device for digital volume tomography according to the state of the art;

FIG. 2 a device for producing a three-dimensional image of an object, with which a beam source as well as a beam detector is led in a first movement and a second movement;

FIG. 3 a representation of the device which corresponds to FIG. 2 and with which the beam source and the beam detector are however not coupled to one another;

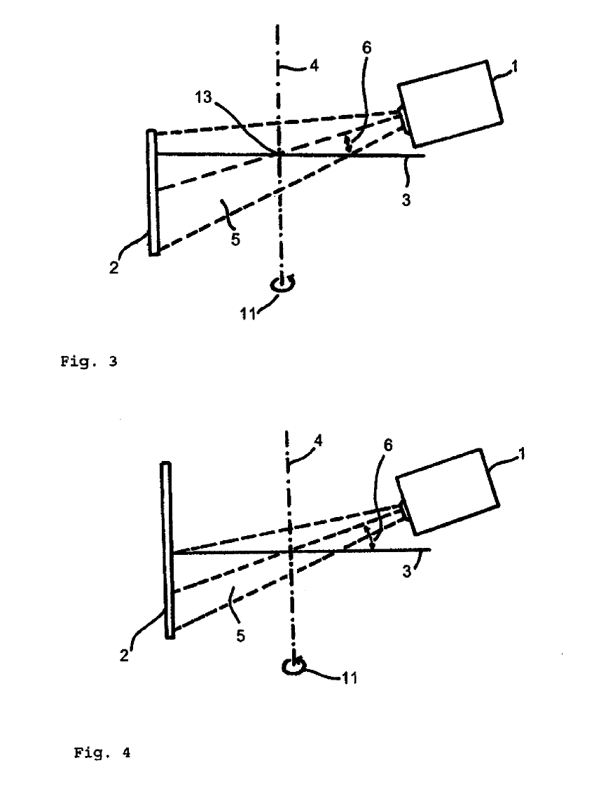

FIG. 4 a representation of the device which corresponds to FIG. 2 and with which only the beam source carries out the second movement;

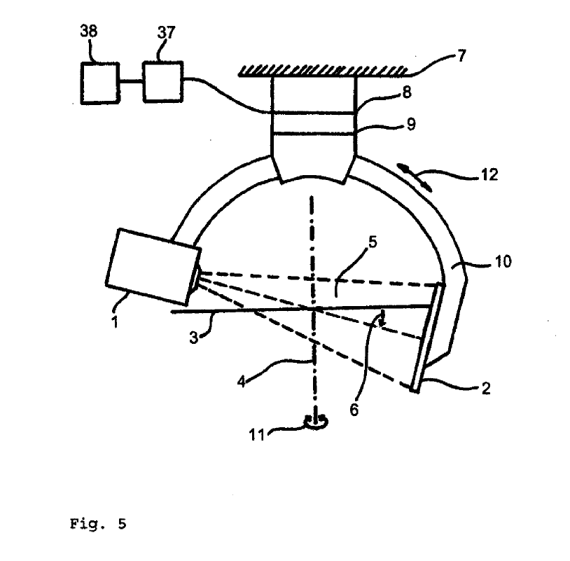

FIG. 5 a lateral view of a tomograph which is moveable in two degrees of freedom;

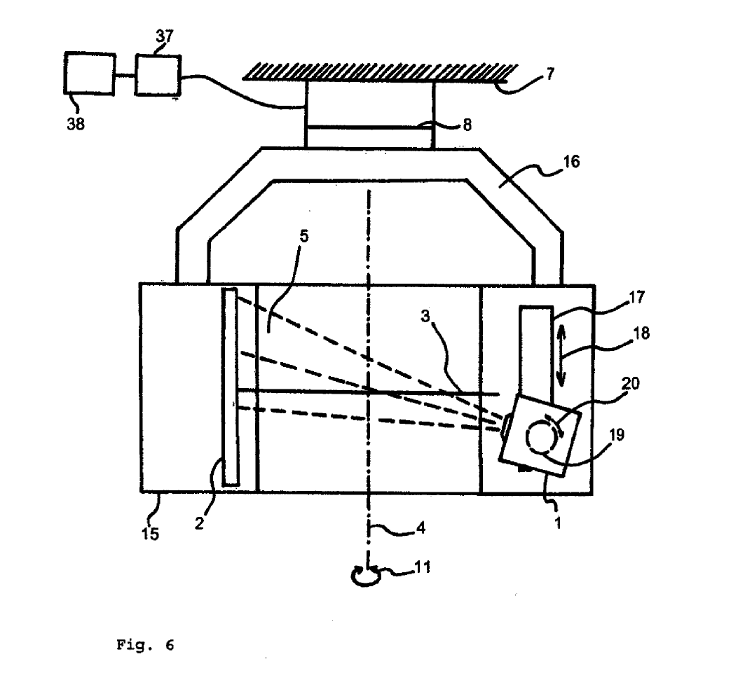

FIG. 6 a lateral view of a tomograph, with which the beam source is movable by way of a linear pivot and a rotation pivot;

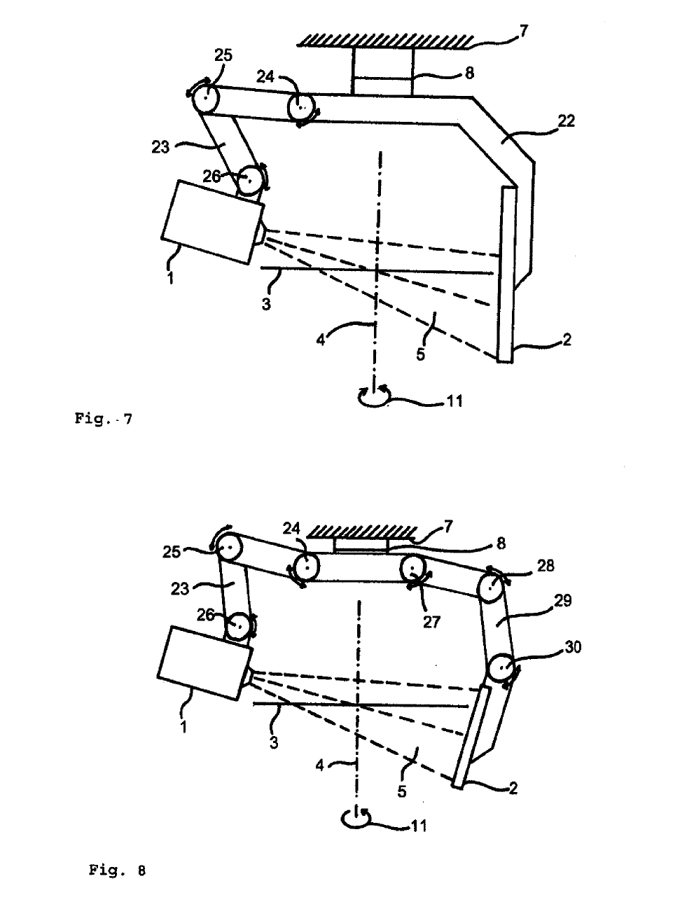

FIG. 7 a lateral view of a tomograph, with which the beam source is fastened on a two-axis robot arm;

FIG. 8 a representation of a tomograph which corresponds to FIG. 7, with which the beam source as well as the beam detector are fastened in each case on a two-axis robot arm;

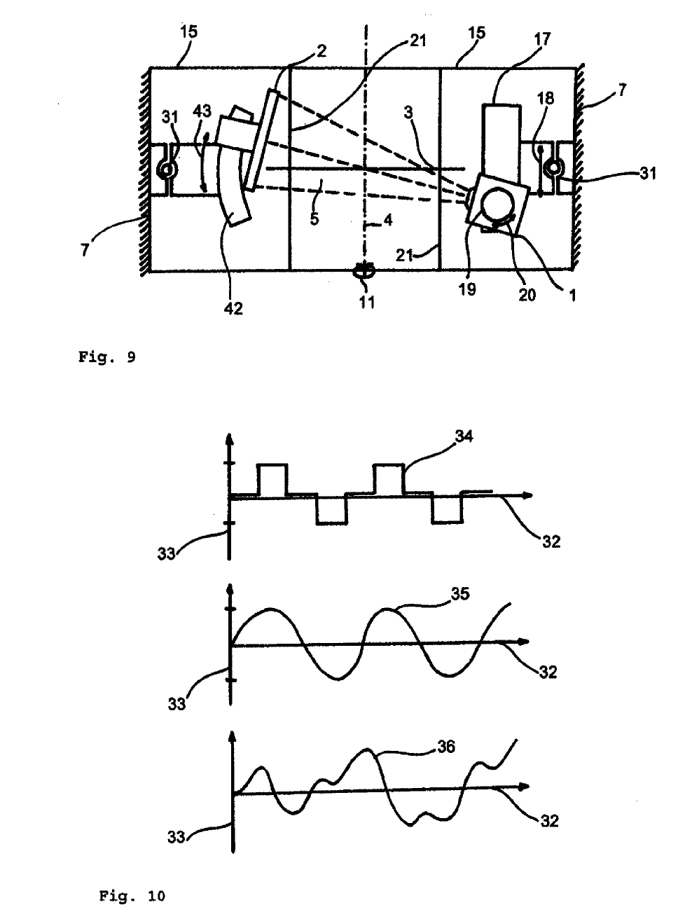

FIG. 9 a lateral view of a tomograph, with which the beam source as well as the beam detector are movable by the linear pivot and the rotation pivot,

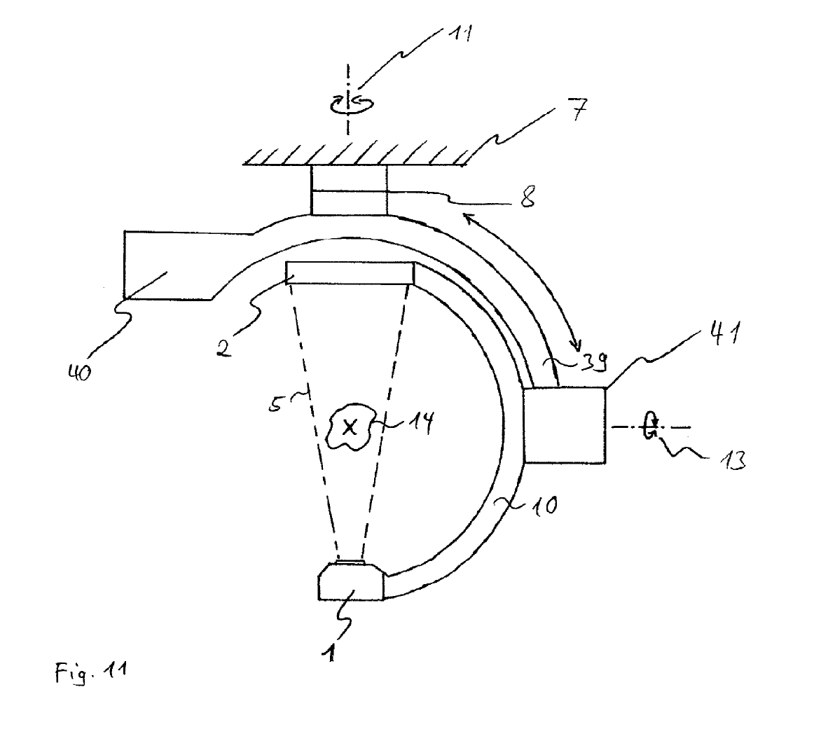

FIG. 10 a temporal course of different types of a second movement and

FIG. 11 a view corresponding to FIG. 5, of further embodiment of a tomograph movable in two degrees of freedom.

FIG. 1 in a lateral view shows a device for digital volume tomography, thus a volume tomograph, with an X-ray source 1 and an X-ray flat detector 2, according to the state of the art. The X-ray source and the X-ray flat detector 2 are arranged lying opposite one another. A plane of a circular path 3, on which the X-ray source 1 and the X-ray flat detector 2 are rotated, runs centrally through the X-ray source 1 and the X-ray flat detector 2. This rotation 11 is effected about the rotation axis 4, wherein the X-ray source 1 and the X-ray flat detector 2 are always located opposite one another during the rotation 11. The X-ray source 1 emits a bundle of X-ray beams 5 which runs in a diverging manner. A middle beam of these X-ray beams 5 runs in the plane of the circular path 3 and is centrally incident on the X-ray flat detector 2 which is at least partly positioned along the beam path. Digital volume tomography in particular is applied in dental medicine, oral surgery, jaw surgery and facial surgery as well as ears-nose-and-throat medicine. Three-dimensional pictures of digital volume tomography are chiefly applied for the diagnostic evaluation by the treating physician.

FIG. 2 shows a lateral view of a device according to the invention, with which the X-ray source 1 and the X-ray flat detector 2 simultaneously and additionally to the rotation movement 11 can execute an inclination angle movement 6, in contrast to the example shown in FIG. 1. Identical features in this and in the following figures are each provided with the same reference numerals. The X-ray source 1 and the X-ray flat detector 2 are coupled to one another in a manner such that they always lie opposite one another during the rotation movement 11 and the inclination angle movement 6. The first movement about a target volume 14 of an object to be imaged is again the rotation movement 11. Additionally, the X-ray source 11 and the X-ray flat detector 2 are raised or lowered with respect to the plane of the circular path 3, by way of the inclination angle movement 6.

The plane of the circular path 3 thus divides a space surrounding the tomograph into two half-spaces, wherein the X-ray source 1 and the X-ray flat detector 2 are led periodically in one of the two half-spaces. The plane of the circular path 3 lies horizontally, so that for example a seated patient can be examined with the arrangement represented in FIG. 2. Thus the X-ray source 1 is temporally located above the horizontally lying plane of the circular path 3. The X-ray flat detector 2 is accordingly located below the mentioned plane in these positions of the X-ray source. The X-ray flat detector 2 runs through positions located above this plane inasmuch as the X-ray source 1 is located below the plane of the circular path 3. If the plane runs vertically the arrangement of the X-ray source 1 and the X-ray flat detector 2 in sections would be located to the left and right of this plane instead of above and below it. In further embodiments, other construction forms of X-ray detectors can also be applied instead of the X-ray flat detector 2. Of course, also other types of radiation could be additionally or alternatively used instead of X-ray radiation, as long as these are suitable as through-beaming radiation.

Projection perspectives of the target volume 14 which would not be accessible with a device according to FIG. 1 are possible by way of the superposition of the circular rotation movement 11 with the inclination angle movement 6 with at least one further mechanical degree of freedom. The circular path 3 with the embodiment example represented in FIG. 2 is a closed circular path 3, i.e. the X-ray source 1 as well as the X-ray flat detector 2 are moved by 360° about the target volume 14, wherein the starting point and the end point of the rotation coincide. In further embodiment examples, the circular movement however can also only encompass a certain angular region, for example only 180°. Likewise, in further embodiment examples, the X-ray source 1 and the X-ray flat detector 2 instead of being led on a circular path can be led on an elliptical path or a path with a freely selectable course. Picture artefacts of an imaging can be reduced by way of the described method, by way of a movement of the configuration of the X-ray source 1 and the X-ray flat detector 2, said movement being superimposed on the circular movement. The path of the inclination angle movement 6 intersects the circular path 3 at regular distances, so that both paths in sections are identical to one another and in sections are different to one another. A resulting path from the path of the inclination angle movement 6 and the circular path 3 thus results from all spatial points which the X-ray source 1 or the X-ray flat detector 2 run through in a temporal sequence. An evaluation from several perspectives is rendered possible by way of the recording of pictures from different positions, which can be achieved by the inclination movement 6 as the second movement.

In the embodiment example represented in FIG. 2, an implementation and a use of at least one further picture recording plane running through an object to be imaged, is sought after by way of the inclination of the configuration of the X-ray source 1 and the X-ray flat detector 2 with respect to a target volume centre, in order to thus achieve an inclination of the object to be imaged, in the picture recordings. Additional projection perspectives are produced by way of this. A distance between the X-ray source 1 and the target volume 14 and which is measured from a point of the facing surface of the X-ray source, said point being closest to the object to be imaged, to the middle point of the target volume 14 along a central beam is constant during the movement of the X-ray source 1. A distance between the X-ray flat detector 2 and the target volume 14 and which is determined in the same manner is likewise constant. In further embodiment examples, further points, for example the geometric centre point of the X-ray source 1, of the X-ray flat detector 2 or of the target volume 14 or their centres of gravity can also be used as reference points for determining the distances. Likewise points on the surface of the X-ray source 1, of the X-ray flat detector 2 or of the target volume 14 can also be used as reference points.

The inclination angle movement 6 in the example which is represented in FIG. 2 sweeps an angular range of between −45° to 45° departing from the plane of the circular path 3, i.e. the inclination angle movement 6 runs above as well as below the mentioned plane. In further embodiment examples, one can of course also envisage a smaller or a larger angular range being passed through with the inclination angle movement 6. Alternatively, the inclination angle movement 6 can also be effected only on one side of the plane of the circular path 3, i.e. for example only above this plane or only below this plane. The inclination angle movement 6 is freely selectable with regard to its course. It can for example be effected sinusoidally or in discrete steps. Typically, the inclination angle movement 6 however is a complete periodic movement, wherein a frequency of the rotation movement 11 is precisely half the magnitude of a frequency of the inclination angle movement 6. Thus two cycles of the inclination angle movement are effected with one cycle (revolution) of the rotation movement. In further embodiment examples, the ratio of the two frequencies to one another can of course also assume other values, for example 1:1 or instead of 1:2 also 2:1. Thus the movement of the X-ray source 1 and of the X-ray flat detector 2 is effected on a virtual spherical surface of a sphere with a radius which corresponds precisely to the distance between the X-ray source 1 and the target volume 14 or between the X-ray flat detector 2 and the target volume 14. The distance between the X-ray source 1 and the target volume 14 and the distance between the X-ray flat detector 2 and the target volume 14 can thereby be equally large or differently large. Typically, the distance between the X-ray source 1 and the target volume 14 is greater than the distance between the X-ray flat detector 2 and the target volume 14. Alternatively, the distance between the X-ray source 1 and the target volume 14 can also be smaller than the distance between the X-ray flat detector 2 and the target volume 14.

With the embodiment example represented in FIG. 2, the speed of the first movement, i.e. of the rotation movement 11, and of the second movement, i.e. of the inclination angle movement 6 are constant. These speeds, i.e. the angular speeds of the movement can of course also be variable at least in sections, in further embodiment examples. Hereby, an angular speed of the second movement at least in sections can be greater than an angular speed of the first movement. In a further embodiment example, a reduction of the angular speed of the rotation movement as the first movement is carried out with an increase of the angular speed of the inclination movement as the second movement.

The X-ray source 1 relative to the target volume 14, as well as the X-ray flat detector 2 by way of the coupling to the X-ray source 1, are moved into several positions, in which a picture is taken in each case, for producing a three-dimensional image of the target volume 14 of the object to be imaged, in the embodiment example represented in FIG. 2 a set of teeth as a target volume of skull 14 as the object to be imaged. Instead of recording exactly one picture in each position, of course a multitude of pictures can be recorded in this position, or also positions, in which firstly no pictures are taken, can be moved to. The three-dimensional image of the target volume 14 is reconstructed from the recorded pictures by way of an evaluation unit which is not represented in FIG. 2, by way of an iterative volume reconstruction method, such as a volume reconstruction algorithm. An adapted back-projection method can also be alternatively or additionally used for computing the three-dimensional image of the target volume. The evaluation unit comprises a computer with an output unit, for example a monitor. The three-dimensional image of the target volume 14 is represented on the output unit, and two-dimensional picture recordings which were made in the moved-to positions can likewise be outputted on the monitor. A transfer of picture data from the X-ray flat detector 2 to the evaluation unit can be effected by way of a cable or also in a wireless manner

A centre point—or more precisely a centre of gravity—of the target volume 14 to be imaged lies in an axis intersection point of the rotation axis 4 with the inclination axis 13. The centre point can however also be given by a geometric centre point resulting from the dimensions of the target volume 14 or an anatomically conspicuous and/or other region of interest, which can be independent of the mass and the geometry of the object, instead of the mass centre of gravity. During the movement of the X-ray source 1 and the X-ray flat detector 2, a surface normal of the mentioned apparatus is always onto the middle point of the target volume 14 and distance between the X-ray source 1 and the middle point as well as a distance between the X-ray flat detector 2 remains constant. In further embodiments, of course at least one of the two distances, but also both distances can be varied during the movements. The rotation axis 4 is perpendicular, i.e. at rights angles to the plane of the circular path 3. Likewise the rotation axis 4 is perpendicular to the inclination axis 13 and typically lies in a plane of the second movement. The inclination axis 13 lies completely in the plane of the circular movement 3.

The generation of the mentioned additional projection perspectives can be realised by the system approaches which are yet described in more detail in the following figures by way of embodiment examples.

A volume tomograph is represented in FIG. 3 in a representation corresponding to FIG. 2, with which the X-ray source 1 and the X-ray flat detector 2 are no longer coupled to one another. Now only the X-ray source 1 is led in the rotation movement 11 about the rotation axis 4 and the inclination angle movement 6 about the inclination axis 13, due to the absent coupling. The X-ray flat detector 2—in the represented embodiment example is a flat detector with a scintillator layer—is displaced with an inclination angle movement 6 and co-executes the rotation movement 11. The X-ray radiation 5 which is emitted by the X-ray source 1 thus hits the X-ray flat detector 2 in a largely oblique manner, thus not orthogonally, in contrast to the orthogonal incidence in the previous figures. However, in further embodiments, one can also envisage the X-ray flat detector 2 likewise undergoing at least the inclination angle movement 6, i.e. not only the X-ray source 1 being displaced by inclination during the rotation movement 11 which the X-ray source 1 as well as the X-ray flat detector 2 undergo. The X-ray flat detector 2 does not need to be displaced beyond the rotation movement 11, but moves counter to the X-ray source 1 during the inclination movement 6. Hereby, the X-ray radiation does not need to hit the X-ray detector 2 orthogonally, thus does not need to be orthogonal to the central beam. Likewise, one can of course also envisage the X-ray flat detector 2 only carrying out the rotation movement 12, but not the inclination angle movement 6.

FIG. 4 in a representation corresponding to FIG. 2 shows a volume tomograph, with which the X-ray source 1 and the X-ray flat detector 2 are likewise not coupled to one another. The X-ray flat detector 2 is however now enlarged accordingly, in order to also be able to capture all X-ray beams 5 which are emitted by the X-ray source 1, due to the enlarged detector surface. The X-ray flat detector 2 and the X-ray source 1 thus undergo the first movement as well as the second movement. Of course, detectors with an arched detector surface are also possible in further embodiments, instead of a flat detector 2.

The digital volume tomograph which is represented in the previous figures can be realised mechanically in different ways and manners.

FIG. 5 in a lateral view shows a rigid connection between the X-ray source 1 and the X-ray flat detector 2 by way of a C-arm 10 as a connecting frame element. The volume tomograph which is represented in FIG. 5 is fastened on a ceiling 7 or on a mount. A driven rotation pivot 8 of the volume tomograph which is connected to a driven inclination pivot 9 attached below the driven rotation pivot 8 is fastened on the ceiling 7. The complete arrangement is connected to the evaluation unit 37. The obtained pictures, two-dimensional as well as three-dimensional are outputted on a monitor 38 which is connected to the evaluation unit 37.

The inclination pivot 9 as well as the C-arm 10, by way of rotation of the driven rotation pivot 8 is rotatable about the rotation axis 4 running centrally through the driven rotation pivot 8 and the driven inclination pivot 9. The driven inclination pivot 9 can guide the C-arm 10 in an inclination movement 12, so that the X-ray source 1 rigidly connected to the C-arm 10 and the X-ray flat detector 2 likewise rigidly connected to the C-arm 10 carry out the inclination angle movement 6, so that the represented volume tomograph has two degrees of freedom, specifically rotating and inclining (tilting). All of the mentioned movements can be carried out in a fully automated manner by the driven rotation pivot 8 and the driven inclination pivot 9 as well as in a manually settable manner. With a fully automatic implementation, a computer program product is stored on the computer as the evaluation unit 37 and this activates the digital volume tomograph according to the command sequence contained in the computer program product.

The movements in the embodiment example represented in FIG. 5 are automated and can be programmed by the user into the evaluation unit 37 which is also used for the control. The positions can however also be moved to in a manual manner by the user.

A further lateral view of an embodiment example of the digital volume tomograph with which the inclination movement 12 of the X-ray source 1 is effected by way of a linear pivot 17 and a rotation pivot 19 is represented in FIG. 6. The driven rotation pivot 8 is again connected to the ceiling 7, but now however holds a fastening frame 16 which in further embodiments can also be an outer-lying radial bearing, at whose end a housing 15 is fastened. The X-ray flat detector 2 is mounted in the housing 15. A flat detector with a smaller surface can also be used in further embodiments, wherein this flat detector is arranged on a pivot which moves the flat detector upwards and downwards. Likewise, the linear pivot 17, on which the X-ray source 1 can be moved upwards and downwards in the vertical direction in a linear movement 18 is likewise arranged on the housing 15 in a manner lying opposite the X-ray flat detector 2. The X-ray source 1 is fastened directly on the linear pivot 17, but however can also be additionally rotated in a rotation movement 20 via the rotation pivot 19, so that the inclination angle 6 is realised by a movement about the rotation pivot 19.

A further possible realisation of the digital volume tomograph is represented in a lateral view in FIG. 7. A base frame 22, to which the X-ray flat detector 2 is rigidly connected is fastened on the driven rotation pivot 8 which in turn is fastened on the ceiling 7. The X-ray source 1 however is connected to a two-axis robot arm 23 which comprises two rotation joints 24 and 25. The robot arm 23 is connected to the base frame 22 via the rotation joint 24. The X-ray source 1 is connected to the robot arm 23 via a further rotation joint 26. The rotation joint 26 is hereby arranged directly on the X-ray source 1, so that a torque where possible can be kept to minimum given a movement of the rotation source 1, and no further parts of the robot arm 23 need to be moved. A distance between the rotation joint 24 and the rotation joint 25 correspond precisely to a distance between the rotation joint 25 and the rotation joint 26. In further embodiments, this distance however can also be larger or smaller than described. The at least two-axis robot arm 23 permits only the inclination movement 12, whilst the rotation movement 20 remains superimposed by way of moving the complete arrangement of the X-ray source 1, the X-ray flat detector 2, the base frame 22 and the robot arm 23.

FIG. 8 represents a further development of the digital volume tomograph which is represented in FIG. 7. The X-ray flat detector 2 is now likewise fastened on an at least two-axis robot arm 29. This robot arm 29 likewise comprises two rotation joints 27 and 28. The X-ray detector 2 is connected to the robot arm 29 via a rotation joint 30 which bears directly on the X-ray detector 2. The rotation joints 24, 25, 26, 27, 28 and 30 can be rotated in each case by at least 300°. A distance from the rotation axis 4 to the rotation joint 24 corresponds precisely to the distance from the rotation axis 4 to the rotation joint 27. The robot arm 23 and the robot arm 29 with regard to the arrangement of their rotation joints are generally constructed equally. However, the robot arm 23 and the robot arm 29 can of course also be constructed differently in further embodiments.

FIG. 9 represents a further variant of the digital volume tomography, with which the X-ray source 1 and the X-ray flat detector 2 can be moved via a radial bearing 31 and an associated radial drive. The X-ray source 1 here is movably arranged on the linear pivot 17 and the rotation pivot 19, as already represented in FIG. 6. The linear pivot 17 however lies on the radial bearing 31 and can be moved along this. The radial bearing 31 is connected to a mount 7 and is fastened on this. The X-ray flat detector 2 is likewise displaceably arranged on an arcuate linear pivot 42 by way of a movement 43. The arcuate linear pivot 42 is likewise mounted on one of the radial bearings 31 and can accordingly be moved on this. The X-ray source 1, the rotation pivot 19, the linear pivot 17 and the radial bearing 31 are arranged in the housing 15. The housing parts 21 which lie between the X-ray source 1 and the X-ray flat detector 2 are of a material which is transparent to X-ray radiation. In further embodiments the complete housing 15 can be of a material which is transparent to X-ray radiation. The X-ray flat detector 2 is likewise arranged in a movable manner within the housing 15, on the radial bearing 31. The embodiment represented in FIG. 9 characterises a construction manner which is open at both sides and with which digital volume tomography reduced in metal artefacts can be applied for extensive regions of a human body. The embodiment examples represented in FIGS. 7-9 are also activated via the evaluation unit 37 with the monitor 38 and obtained picture data evaluated.

The arrangements which are represented in FIG. 5-9 are applied in the medical field for digital volume tomography, i.e. in particular with dental-medical examinations or in oral surgery, jaw surgery, facial surgery or ears-nose-and-throat medicine.

Three possible temporal courses of the second movement are represented in FIG. 10. In each case, an inclination angle value in degrees is plotted on an inclination angle value axis 33 over a time axis 32. The time is seconds is plotted on the time axis. The first course 34 characterises a discrete inclination angle course, with which the inclination angle is changed periodically in discrete points. A harmonic course of the inclination angle in a sinusoidal shape is present with the second represented course 35. The third course 36 finally characterises a completely freely selectable course of the inclination angle, for example in the form of a polynomial of the nth degree, wherein “n” characterises a natural number. Hereby, the inclination angle region is not kept constant but is adapted to a geometry of the object to be imaged. Generally, the angular speed of the rotation movement 11 remains constant and the constant rotation movement 11 is superimposed on the inclination angle movement 6. However, in further embodiments, it is also conceivable to variably hold the angular speed of the rotation movement 11. This permits a rapid inclination angle movement 6 to be slowed down by way of an adaptation of the angular speed of the rotation movement 11 and thus the increase of a mechanically dependent accuracy by way of reduced acceleration forces.

A further embodiment of a tomograph which is movable in two degrees of freedom is shown in FIG. 11 in a lateral view corresponding to FIG. 5. The tomograph in turn is fastened on the ceiling 7 or a mount with the rotation pivot 8 and can be rotated about the rotation pivot 8 in the rotation movement 11. A guide 39 which is in the shape of a circular arc of 120° is attached on the rotation pivot 8. A counterweight or compensation weight 40 is arranged at a left end of the guide 39. An inclination joint 41 can be guided between the end of the guide 39 which lies opposite the compensation weight 40, and the rotation pivot 8. This inclination joint 41 in the embodiment example represented in FIG. 11 is at an angle of 90° to the rotation pivot 8, but any angle between 0° and 90° can be set. The C-arm 10 is arranged on the inclination joint 14 and can be rotated about the inclination axis 13. The X-ray flat detector 2 and the X-ray source 1 which can be rotated and tilted about the target volume 14 are attached at ends of the C-arm 10 which are opposite one another.

Features of the different embodiments which are disclosed only in the individual embodiment examples can be combined with one another and claimed individually.

### LIST OF REFERENCE NUMERALS

1 X-ray source

2 X-ray flat detector

3 circular path

4 rotation axis of the circular path

5 X-ray radiation

6 inclination angle movement

7 ceiling/mount

8 rotation pivot

9 inclination pivot

10 C-arm

11 rotation movement

12 inclination movement

13 inclination axis

14 target volume

15 housing

16 fastening frame

17 linear pivot

18 linear movement

19 rotation pivot

20 rotation movement

21 housing transparent to X-ray radiation

22 base frame

23 robot arm

24 rotation joint

25 rotation joint

26 rotation joint

27 rotation joint

28 rotation joint

29 robot arm

30 rotation joint

31 radial bearing

32 time axis

33 inclination angle value axis

34 discrete inclination angle course

35 harmonic inclination angle course

36 free inclination angle course

37 evaluation unit

38 monitor

39 guide

40 compensation joint

41 inclination joint

42 arcuate liner pivot

43 movement

1. A method for producing a three-dimensional image of an object by way of an imaging device, the method comprising:
obtaining or providing or using a movable beam source,
obtaining or providing or using a beam detector and
obtaining or providing or using an evaluation unit,
wherein the beam source for picture recording is moved relative to the object to be imaged, on a first path lying in a first plane, in a first movement into several positions, in which at least one picture is recorded in each case,
wherein the three-dimensional image is reconstructed from the recorded pictures by way of the evaluation unit,
wherein for reducing an artefact in the image and which is produced by a shadowing, the beam source simultaneously to the first movement carries out a second movement relative to the object to be imaged, on a second path which at least in sections is different to the first path, and
wherein the first movement and the second movement superimpose and a distance between the beam source and the object to be imaged during the superposition of the first movement and the second movement is constant.

2. The method according to claim 1, wherein at least a part of the object to be imaged, preferably a centre point of the object to be imaged, lies in an axis intersection point of a first axis, about which the first movement is effected, and a second axis, about which the second movement is effected, wherein the first axis is perpendicular to the first plane.

3. The method according to claim 2, wherein the first axis and the second axis are perpendicular to one another.

4. The method according to claim 1, wherein the second movement at least in sections is effected in one of two half-spaces defined by the first plane, wherein the second movement at least in sections is effected in both half-spaces and/or the second movement at least in sections is a periodic movement.

5. The method according to claim 1, wherein the first movement is a circular movement, an elliptical movement or a freely selectable movement and/or the second movement is an inclination movement, wherein at least the beam source is moved in the circular movement on a circular path about at least 90° and/or at least the beam source in the inclination movement is inclined with respect to the plane of the circular movement by maximally between 1° and 45°.

6. The method according to claim 1, wherein a speed of the first movement and/or a speed of the second movement is constant or variable in sections.

7. The method according to claim 1, wherein a distance between the beam source and the object to be imaged is smaller or equal to a distance between the beam detector and the object to be imaged.

8. The method according to claim 1, wherein the beam source revolves around the object to be imaged during the recording.

9. The method according to claim 1, wherein only the beam source carries out the first movement and the second movement and the beam detector only the first movement, during the recording.

10. The method according to claim 1, wherein the first movement and the second movement are periodic movements, wherein the second movement has a higher frequency than the first movement.

11. The method according to claim 1, wherein an angular speed of the second movement at least in sections is greater than an angular speed of the first movement.

12. The method according to claim 1, wherein a reduction of the angular speed of the first movement is effected given an increase of the angular speed of the second movement.

13. The method according to claim 1, wherein the reconstructed three-dimensional image and/or the recorded pictures are outputted by the evaluation unit.

14. A device for producing a three-dimensional image of an object, said device comprising:
a beam source led about the object to be imaged, in a first movement on a first path lying in a first plane,
a beam detector, and
an evaluation unit,
wherein the beam source simultaneously to the first movement can be moved relative to the object to be imaged, by way of a second movement on a second path, for reducing an artefact of the obtained image which is produced by a shadowing, and
wherein the first movement and the second movement superimpose and a distance between the beam source and the object to be imaged is constant during the superposition of the first movement and the second movement.

15. The device according to claim 14,
wherein the beam detector is movable,
wherein the beam detector is movable on the first path and/or the second path and/or the beam source and/or the beam detector are capable of being inclined with respect to the plane of the first movement for carrying out the second movement, and
wherein preferably the beam source and the beam detector are configured to always be located lying opposite one another.

16. The device according to claim 14,
wherein the first path and/or the second path of the beam source and/or of the beam detector are settable by way of a linear pivot, a rotation pivot and/or a motorised joint arm, and
wherein the motorised joint arm includes a robot arm which comprises at least three joints.

17. The device according to claim 14, wherein the device is an X-ray device with an X-ray source as a beam source and with an X-ray detector as a beam detector.

18. A computer program product, containing a command sequence stored on a machine-readable carrier, for carrying out the method and/or for activating the device according to claim 14, when the computer program product runs on a computation unit, including instructions for:
obtaining or providing or using a movable beam source,
obtaining or providing or using a beam detector and
obtaining or providing or using an evaluation unit,
wherein the beam source for picture recording is moved relative to the object to be imaged, on a first path lying in a first plane, in a first movement into several positions, in which at least one picture is recorded in each case,
wherein the three-dimensional image is reconstructed from the recorded pictures by way of the evaluation unit
wherein for reducing an artefact in the image and which is produced by a shadowing, the beam source-simultaneously to the first movement carries out a second movement relative to the object to be imaged, on a second path which at least in sections is different to the first path, and
wherein the first movement and the second movement superimpose and a distance between the beam source and the object to be imaged during the superposition of the first movement and the second movement is constant.

19. The method according to claim 1, performed in the framework of digital volume tomography, for dental medicine, oral surgery, jaw surgery, facial surgery, ears-nose-and-throat medicine and/or destruction-free industrial imaging.
